(19) United States
(12) Patent Application Publication
(10) Pub. No.: US 2016/0296165 A1
(43) Pub. Date: Oct. 13, 2016

(54) SCREENING SYSTEM FOR ASSESSING SLEEP ABNORMALITIES

(71) Applicant: Dymedix Corporation, Shoreview, MN (US)

(72) Inventors: James P. Moore, Bloomington, MN (US); Todd M. Eiken, Lindstrom, MN (US)

(21) Appl. No.: 14/683,509

(22) Filed: Apr. 10, 2015

(51) Int. Cl. A61B 5/00; A61B 5/087

(52) U.S. Cl. CPC A61B 5/4818; A61B 5/087; A61B 5/7282; A61B 5/7278; A61B 5/682; A61B 2560/0475; A61B 2505/07

(57) ABSTRACT

A home screening method for assessing whether a person is in need of a full sleep study in a sleep lab or is in need of immediate treatment for apnea and/or hypopnea. In carrying out the method, a screening service company provides a customer with a PVDF air flow sensor and an electronics module that connects to the sensor for filtering the sensor analog waveform due to temperature changes upon inspiration and expiration and due to mechanical stress due to snoring. The filtered signals are converted to a digital representation and stored during a period of sleep. The electronics module is then returned to the screening service where the stored information is analyzed in accordance with a program run on a host computer to identify the type, frequency, duration of detected events. The program further generates a report with recommendations for further action.

### CROSS-REFERENCED TO RELATED APPLICATIONS

Not applicable

### STATEMENT REGARDING FEDERALLY SPONSORED RESEARCH OR DEVELOPMENT

Not applicable

### BACKGROUND OF THE INVENTION

I. Field of the Invention

This invention relates generally to a home screening method of assessing whether a person is a candidate for a full sleep study or for immediate treatment using a CPAP device.

II. Discussion of the Prior Art

Humans spend almost 30% of their lives sleeping. Since the 1970's, physicians have begun to recognize many of the detrimental consequences of sleep disturbances produced by abnormal breathing patterns, or sleep-disordered breathing (SDB). Sleep apnea and other sleep-related breathing disorders constitute the greatest number of sleep disorders seen by sleep medicine, pulmonary, and general practitioners in the outpatient setting. SDB has been associated with considerable morbidity.

SDB comprises a wide spectrum of sleep-related breathing abnormalities; those related to increased upper airway resistance include snoring, upper airway resistance syndrome (UARS), and obstructive sleep apnea-hypopnea syndrome (OSAHS). Many clinicians regard SDB as a spectrum of diseases. This concept suggests that a person who snores may be exhibiting the first manifestation of SDB and that snoring should not be viewed as normal. A patient can move gradually through the continuum, for example, with weight gain and eventual development of Pickwickian syndrome or with alcohol or sedative use, which can cause a person who snores to turn into a snorer with obstructive sleep apnea (OSA). This concept has support from experimental studies showing increasing airway collapsibility during sleep with progression from normal, snoring, UARS, and OSA. Continuous positive airway pressure (CPAP) can effectively treat apnea, but the patient may be left with continued residual UARS or snoring. Therefore, the clinician must recognize that this disease entity represents a continuum and that patients can continue to suffer from symptoms caused by one aspect of SDB while being treated for another aspect.

Snoring is one of the most common aspects of SDB and has been described throughout history. In the past, snoring generally had been considered a social nuisance with no consequences for the snorer, only for the suffering bed partner. After sleep apnea syndrome was recognized, snoring began to be viewed as an important clinical symptom. Although it is by far the most common symptom of sleep apnea and is usually the main reason for a patient visit, patients by themselves are generally not disturbed by the snoring. Instead, it is at the prompting of the bed partner, whose sleep is disrupted due to snoring that the patient sees a physician. Of course, not all patients who snore have sleep apnea.

Snoring is a result of the changes in the configuration and properties of the upper airway (from the nasopharynx to the laryngopharynx) that occurs during sleep. Any membranous portion of the airway that lacks cartilaginous support, including the soft palate, uvula, and the pharyngeal walls, can produce this sound. Snoring is usually an inspiratory sound, but it can also occur in expiration. Snoring can occur during any stage of sleep but is more common during stages 2, 3, and 4. This is because airway elasticity and muscle tone due to sympathetic activity and neural output to the upper airway walls are different during rapid eye movement (REM) and non-REM sleep. Multiple predisposing factors can lead to a snoring abnormality, including age (middle or advanced), obesity, weight gain, body posture, use of alcohol and muscle relaxants, retrognathia, nasal blockage, development of asthma, and smoking.

A primary snorer is usually asymptomatic and does not suffer from cardiovascular disease. Snoring in this population is usually an annoyance to the bed partner, and the snorer might deny any symptoms of daytime somnolence or difficulty with concentration. In contrast, snoring also can occur in conjunction with a disordered sleep pattern and may be associated with a range of symptoms, including overt OSAHS.

A complete history and careful physical examination are paramount in assessing whether sleep apnea is present in a patient with snoring symptoms. The history and examination results also guide the clinician in deciding whether a nocturnal polysomnogram is necessary and in determining appropriate treatment.

The two main studies usually used to evaluate snoring are nocturnal polysomnography and an airway assessment. In a position statement, the American College of Chest Physicians and the Association of Sleep Disorders Centers have declared that only snorers suspected of having sleep apnea syndrome should undergo polysomnography. The American Thoracic Society has declared in its position statement that snoring alone is not an indication for a sleep study.

In symptomatic snorers with daytime somnolence, reduced performance, reduced attention, drowsy driving and tiredness, a full nocturnal polysomnogram are needed to establish a diagnosis of sleep apnea or UARS. Nocturnal polysomnography with a recording of sleep architecture and arousals is necessary.

Polysomnography remains the gold standard for diagnosing SDB. A complete polysomnography is often termed a full sleep study. Sleep is recorded from a number of electrophysiologic signals, as well as from breathing and limb movement electrodes. This includes an electroencephalogram (EEG) with two leads, electromyography, electro-oculography, respiratory signals from airflow measurements from nasal pressure, nasal temperature, expired carbon dioxide, ventilation from thoracoabdominal movements or nasal pressure, oxygenation levels, and possibly esophageal balloon pressures. Other signals include an electrocardiogram tracing during sleep, pulse rate, position, esophageal pH, and video recording. A detailed airway assessment of upper airway volume and area is not performed routinely because it does not predict a successful surgical outcome in a non-apneic snorer. If surgery is being considered, further radiographic imaging can provide an airway assessment and may include cephalometric measurements, computed tomography, or magnetic resonance imaging.

Upper airway resistance syndrome or UARS can cause symptoms similar to those found in obstructive sleep apnea or OSA, yet this syndrome is considerably different due to the absence of oxygen desaturation found during sleep studies. UARS was a term first applied to patients who were found to have excessive daytime sleepiness without a clear cause on a multiple sleep latency test, which was further documented by an overnight polysomnogram. These patients were often said to have idiopathic hypersomnia. After many of these patients were further tested with invasive polysomnography, they were found to have increased upper airway resistance. Resistance manifested as increased negative esophageal inspiratory pressure.

UARS is characterized by repeated arousals, due to resistance to airflow in the upper airway, that lead to excessive daytime sleepiness and fatigue. Snoring has been noted to be present in association with these brief arousals, but snoring is not necessary for identification of UARS. UARS events are noted to be typically short; 1 to 3 breaths in duration. These events have been termed respiratory effort-related arousals (RERAs). In UARS, unlike in obstructive sleep apnea hypopnea syndrome or OSAHS, there is no evidence of oxygen desaturation. For the measurement criteria to be classified as a RERA, there must be a pattern of progressively increased negative esophageal pressure that is terminated by a sudden change in the pressure to a less-negative level and a sleep arousal. Furthermore, the event must last 10 seconds or longer.

It has been demonstrated that many non-apneic patients show a reduction in cross-sectional area of the pharynx during sleep. Reduction in airway area is sufficient to avoid hypopnea and apnea but enough to increase upper airway resistance. These patients also have increased airway collapsibility due to abnormal anatomy. Patients with UARS suffer from increased airway resistance, which leads to arousal episodes and ultimately to excessive daytime sleepiness. Nasal airway anatomic issues (i.e., deviated septum, inferior turbinate hypertrophy, nasal valve collapse, or any combination of these) have been associated with UARS.

Clinical presentation of UARS can be varied. The cardinal symptoms of UARS are fatigue and excessive daytime sleepiness. Some patients also complain of difficulty with concentration, morning headaches, impotence, and difficulty with sleep onset and sleep maintenance (insomnia). Snoring is not a necessary feature of this syndrome because the upper airway resistance is due to a partial decrease in airway cross-sectional area and the airway walls do not have to vibrate to produce a snoring sound. It is now increasingly recognized that the clinical features seen in UARS overlap with functional somatic syndromes such as chronic fatigue syndrome, fibromyalgia, irritable bowel syndrome (IBS), chronic headache, and temporomandibular joint (TMJ) syndrome. Based on the signs and symptoms alone, it can be difficult to distinguish the patients with UARS from those with mild OSAHS.

The diagnosis of UARS requires a high degree of clinical suspicion. Diagnosis of UARS requires symptoms (excessive daytime somnolence, fragmented sleep, fatigue), anatomic features consistent with upper airway narrowing, and supportive PSG findings.

UARS is present only if there are documented elevations in upper airway resistance, sleep fragmentation, and daytime dysfunction or excessive daytime sleepiness. A low respiratory disturbance index (RDI) is also needed to distinguish UARS from OSAHS. The elevated EEG arousal index related to increased respiratory efforts is the specific measurement that distinguishes UARS from idiopathic hypersomnolence. The clinical complaint of snoring (including crescendo snoring), increase in snoring intensity before EEG arousals, and clinical improvement with a short-term trial of nasal CPAP can be regarded as supporting a diagnosis of UARS.

The diagnosis of UARS requires full polysomnography. Although measurements of upper airway resistance were first used, based on the original definition of UARS, substitute measurements of effort and ventilation may be used as long as there is no evidence of hypopnea or apnea. A normal apnea-hypopnea index (AHI) of less than 5 events per hour of sleep should be seen on the polysomnograph. Additionally, EEG arousals should occur at a rate of more than 10 per hour of sleep and must be associated with increased respiratory effort (usually made by nocturnal esophageal pressure monitoring). Studies have shown an association of alpha-delta sleep pattern in the EEG of patients with UARS. Alpha-delta pattern is a nonspecific EEG finding in which there is intrusion of wake alpha pattern into the deep, slow-wave sleep. This is also seen in some functional somatic syndromes listed above, but is not a feature of OSAHS.

The measurement of esophageal pressure is the gold standard for measuring respiratory effort and is the only consistent measurement reported for the diagnosis of UARS. Current literature supports that esophageal pressures more negative than 10 cm H₂O are abnormal. Substitute measurements can include inductive plethysmography, strain gauges, oronasal temperature measurements, nasal pressures, and the carbon monoxide levels in exhaled gas. Arousals are documented from the EEG tracings and electromyography.

OSAHS was identified as a distinct entity only in 1999, despite being present for many years. Evolving from the historical accounts of sleep apnea to the present day, the most significant development in the diagnosis of sleep-disordered breathing was the publication of the American Academy of Sleep Medicine (AASM) report on recommendations for syndrome definition and measurement techniques in clinical research. Within this report, the older term obstructive sleep apnea was appropriately changed to the newer term obstructive sleep apnea hypopnea syndrome. The complications and potential consequences of OSAHS include increased risks of hypertension, cardiovascular events, as well as cerebrovascular events. OSAHS is also associated with an increase in the rate and severity of motor vehicle accidents, increased healthcare utilization, reduction of work performance, and occupational injuries. OSAHS affects not only the health of the sufferer, but also the bed partner's sleep state.

OSAHS is characterized by recurrent episodes of partial or complete airway obstruction during sleep due to repetitive obstruction of the upper airway, necessitating recurrent awakenings or arousals to re-establish airway patency, often with oxygen desaturation. This airway obstruction or partial obstruction manifests in a reduction in airflow, termed hypopnea, or in a complete cessation of airflow, terms apnea, despite ongoing inspiratory efforts. Hypopnea is defined in adults as a 10-second event during which there is continued breathing, but in which ventilation during sleep is reduced by at least 50% from baseline. Apnea is total cessation of airflow for at least 10 seconds. Apnea can be obstructive, central, or mixed. Obstructive apnea is more common and is defined as cessation of airflow, but with continued respiratory effort, whereas central apnea is a state in which airflow and respiratory effort are both absent. Mixed apnea is recognized by a lack of respiratory effort during initial apnea period followed by gradually increasing effort against an obstructed upper airway. These events are thought to be related pathophysiologically to obstructive apnea. Hypopnea can produce clinical sequelae similar to those of apnea, but in general, apnea is associated with a greater fall in oxygen saturation.

For sleep-disordered breathing to be diagnosed as OSAHS, the patient must have at least 5 obstructed breathing events per hour (or 30 events per 6 hours of sleep) on an overnight polysomnogram. These events can be a combination of obstructive apnea and hypopnea (for the determination of an apnea-hypopnea index or AHI) and additional inclusion of the respiratory effort-related arousals (for the determination of the respiratory disturbance index or RDI). The patient must also have either excessive daytime sleepiness or at least two of choking or gasping from sleep, recurrent awakenings from sleep, feeling unrefreshed after sleep, daytime fatigue, or poor concentration. This second group of signs and symptoms must not be better explained by other factors.

The AHI is the number of apneas plus hypopneas per hour of sleep. This index has now become the standard by which to define and quantify the severity of OSAHS. An AHI of more than 5 events per hour indicates possible OSAHS. As the AHI increases, the severity of apnea increases.

OSAHS occurs due to a narrowing of the upper airway during sleep. The site of the narrowing is usually at the level of the pharynx. Airway occlusion is noted to be limited to inspiration, which exerts negative pharyngeal pressure and reduces the tone of the pharyngeal dilator muscles. This theory remains the cornerstone of understanding OSAHS. During REM sleep, there is a further decrease in tone and activity of the pharyngeal dilator muscles causing longer and more distinct apnea and hypopnea events.

Upper airway size in OSAHS patients is smaller than in normal subjects, as assessed by CT scan and resistance measurements. Patients with OSAHS also have been noted to have a more elliptical upper airway shape than normal subjects, but this may be due to increased body mass as well. The difference in airway size in OSAHS patients is due to fat deposition and facial bone structure. Obese patients with OSAHS have fat deposits lateral to the pharynx. Although this fat deposit might not be substantial, it can predispose patients to OSAHS.

Genetics might play an important role in the pathophysiology of OSAHS. The disorder is more common among family members suffering from OSAHS than in the general population. This relation seems to be independent of familial obesity tendencies. There is an increase in familial susceptibility with an increase in number of affected relatives.

Because of many of the symptoms of OSA are nonspecific, the clinician needs to have a high index of clinical suspicion to make the diagnosis of OSAHS. The differential diagnosis for OSAHS should include primary snoring, chronic hypoventilation syndrome central sleep apnea, and Cheyne-Stokes respiration. The other causes of sleepiness that need to be distinguished from OSAHS are narcolepsy, idiopathic hypersomnia, insufficient sleep, and periodic limb movement disorder.

Patients suspected to have OSAHS should undergo an overnight polysomnogram. Due to night-to-night variability in mild cases of the disorder, the diagnosis can be missed. Therefore, a negative first-night test is insufficient to rule out OSAHS in a patient in whom there is clinical reason to suspect the disease.

Many other types of sleep studies are available, with varying settings and parameters measured. A complete level I study is performed in the laboratory; partial and limited studies can be conducted in the home. However, the AASM suggests that standard polysomnography is the accepted test for diagnosing and determining the severity and treatment of OSA. The AASM task force recommends that portable monitoring is an acceptable alternative in patients at high risk for OSAHS without a coexisting medical or sleep disorder. Monitoring should be done in conjunction with a comprehensive sleep medicine evaluation. Portable monitoring can be performed in a patient who cannot be safely transported for laboratory polysomnogram, in whom initiation of treatment is urgent and a standard polysomnography is not readily available, or in whom follow-up studies are needed to evaluate response to therapy.

Adequate treatment of OSAHS results in improvement of symptoms and can alter morbidity and mortality outcomes. Current therapies in the treatment of sleep apnea are intended to widen the pharyngeal airway and make it less apt to collapse, or to pneumatically splint the airway open using CPAP. CPAP therapy is very effective in eliminating pharyngeal collapse, improving overall symptoms, and reducing cardiovascular sequelae, making it the treatment of choice for OSAHS. Although CPAP is the mainstay of therapy for OSAHS, there are other types of positive airway pressure therapies available: bi-level positive airway pressure (bi-level PAP), auto-PAP (APAP), and expiratory pressure relief devices. Bi-level PAP allows the clinician to set different pressures for inspiratory and expiratory breaths. This may be beneficial for patients who occasionally complain of feeling excessive air pressure or of having the sensation of exhaling against positive pressure. The routine use of bi-level PAP has not been shown to increase compliance, but in patients who have high CPAP requirements, bi-level PAP may be a more comfortable option.

Since the 1970's, physicians have begun to recognize many of the detrimental consequences of sleep disturbances produced by abnormal breathing patterns, i.e. SDB. Sleep apnea and other sleep-related breathing disorders constitute the greatest number of sleep disorders seen by sleep medicine, pulmonary and general practitioners in the outpatient setting. Approximately forty-two million American adults have SDB while one-in-five adults exhibit mild obstructive sleep apnea (OSA). One in fifteen adults has moderate to severe OSA. It is estimated that 75% of severe SDB cases remain undiagnosed.

SDB comprises a wide spectrum of sleep-related abnormalities. Many relate to increased upper airway resistance including snoring, sleep apnea and sleep hypopnea.

During the preceding five years, many health insurance providers have begun requiring home sleep testing as a prerequisite to a full examination in a sleep lab environment where multiple sensors and a polysomnograph machine are used to diagnose the SDB and its severity. The test results are then provided to a physician qualified to provide remedial treatment to the subject. Given the typical cost of an in-sleep-lab testing, it makes economic sense to perform a much lower cost HST as a screening tool to determine whether a subject requires the furthermore expensive testing conducted in a sleep lab.

In a full sleep study, a number of electrophysiologic signals, as well as from breathing and limb movement electrodes electroencephalogram and respiratory signals from airflow measurements from nasal pressure and nasal temperature sensors and respiratory effort from abdominal and chest belts are obtained.

Because of the relatively high cost of full sleep studies in hospitals and clinics to assess SDB, a need exists for a low cost method for home screening of subjects to determine whether such a full sleep study is required and, moreover, whether a subject is in immediate need of a CPAP device to address more severe apneas or hypopneas. It is the object of the present invention to provide such a method.

### SUMMARY OF THE INVENTION

The present invention provides a method for screening persons for sleep-disordered breathing in which a screening test facility initially provides to a person to be screened a PVDF respiratory air flow sensor and an electronics module which when coupled to the sensor with the sensor located on a person's upper lip is adapted to record and store signals relating to respiratory activity and snoring. Before sending the electronics module to the person to be screened, the electronics module is first preloaded with that person's demographic information. Following a period of sleep in which the sensor and the electronics module are being worn by the test subject, the electronics module is returned to the screening test facility where the person's demographic information and the information derived from the sensor are downloaded into a host computer at the screening test facility. A program is executed on the host computer for analyzing the sensor-derived information to identify the onset, duration and type of sleep-disordered breathing events and separately logging the type of sleep-disordered breathing events encountered. The host computer then prepares a report identifying, inter alia, total apnea events, total hypopnea events, total unspecified sleep-disordered breathing events, and total snoring events during a calculated total analysis time.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which:

FIG. 1 shows a subject equipped with a pyro/piezo sensor electrically coupled to an electronics module worn by the subject in the course of a home screening for sleep disordered breathing (SDB);

FIG. 2 is a block diagram of the system for carrying out the method of the present invention;

FIG. 3 is a waveform helpful in understanding the software for transforming the sensor-derived signals into meaningful data whereby the SDB can be analyzed; and

FIG. 4 is a snore waveform.

### DESCRIPTION OF THE PREFERRED EMBODIMENT

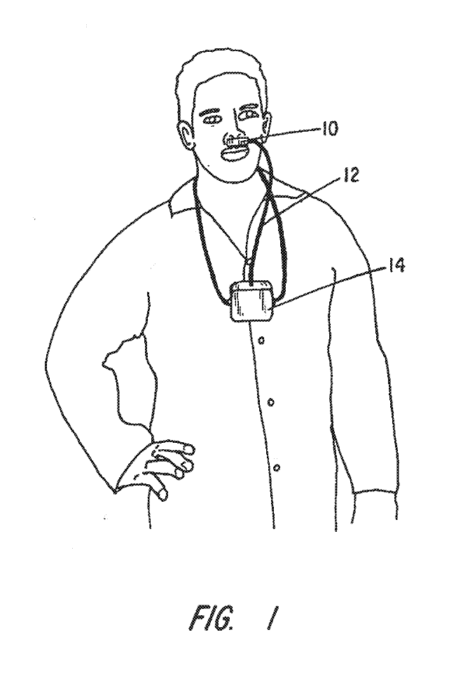

Referring first to FIG. 1, there is shown an illustrated man equipped with a pyro/piezo sensor 10 adhesively mounted on his upper lip where it is exposed to respiratory airflow and vibration due to snoring. The sensor is preferably a PVDF sensor, as described in U.S. Pat. No. 6,485,432, which is assigned to the assignee of the present invention, although U.S. Pat. Nos. 6,491,642 and 8,147,420 also disclose suitable sensors for use with the present invention.

The sensor 10 is shown as being connected by electrical leads 12 to an electronics module 14 shown as being worn by the man who is to undergo a home sleep screening test. The electronics module 14 is shown as being suspended from a lanyard 16 worn about the person's neck.

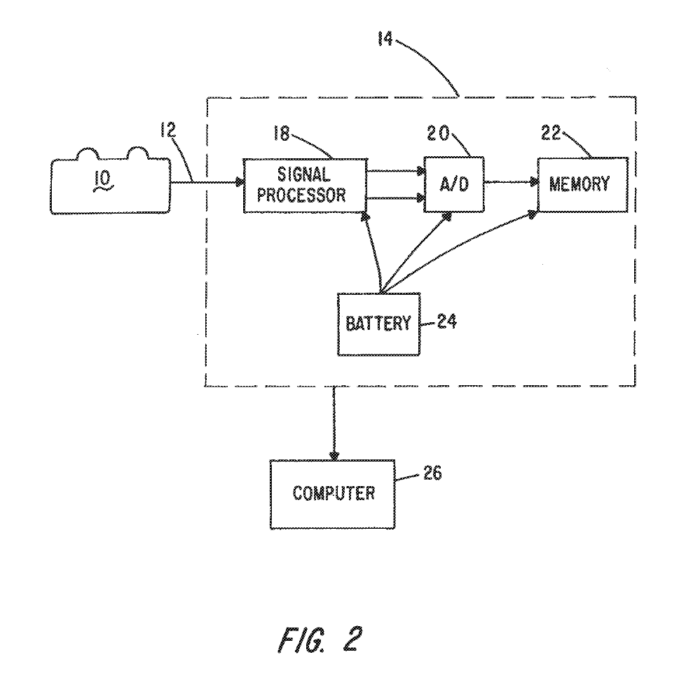

FIG. 2 is a block diagram of the system for carrying out the method of the present invention. As represented there, the pyro/piezo sensor 10 is coupled by leads 12 to the electronics module 14. The module 14 comprises a signal processing circuit 18 connected to deliver its analog signal output to an A/D converter 20 whose digital output is adapted to be stored in a memory device 22. A rechargeable battery 24 provides operating voltages to the signal processor 18, the A/D converter 20 and the memory module 22.

The signal processor 18 is described in U.S. Pat. No. 6,702,755 assigned to applicants' assignee and which is hereby incorporated by reference. As described therein, it functions to amplify and filter the raw signal from the PVDF sensor 10 to yield a first waveform corresponding to temperature variations as respiratory air is made to impinge on the sensor and a second waveform due to vibration induced by snoring.

While the A/D converter 20 and the memory 22 are depicted as separate functional blocks in FIG. 2, those skilled in the electronics arts will recognize that there are any number of commercially available integrated circuits for implementing this functionality. As only one example, a DAC0808 D/A converter may be used as an input to an Intel 8085 microprocessor which includes a 64 KB data memory.

As further indicated by FIG. 2, the electronics module 14 is adapted to be operatively coupled to a computer 26 whereby the data stored in the memory 22 may be read out for analysis. As will be explained in greater detail when the program executed by computer 26 is described, information entered into the computer 26 may also be stored in the memory 22.

In accordance with the method of the present invention, a company (test sponsor) offering home sleep screening services will have a plurality of electronic modules 14 in inventory so that tests can be administered to a large number of individuals. Thus, as an initial step when a home sleep study is ordered, the test sponsor will initialize one of the devices 14 by entering demographic information from the sponsor's computer 26 into the memory 22 of the device 14. This will typically comprise the name of the person to be tested, a file number or other ID code, the person's contact information. At the same time, a check will be made of the battery status. The device 14 and a few PVDF sensor strips, along with the use instructions, will be delivered to the person to be tested.

At bed time, the person will apply the sensor 10 per instructions and the act of plugging the lead 12 into the module 14 will coupled power from the battery 24 to the circuitry within the module 14. The electronics module will then begin recording, in a digital format, the respiration waveforms due to temperature shifts and pressure changes sensed by the PVDF sensor 10 as the person inhales and exhales. In a second channel, the waveforms due to stress fluctuations on the sensor due to snore vibrations will also be digitized and stored in the memory 22.

At the conclusion of a satisfactory period of sleep, the device 14 will be returned to the test sponsor for download of the stored data into its host computer 26 for analysis in accordance with the programmed steps described below.

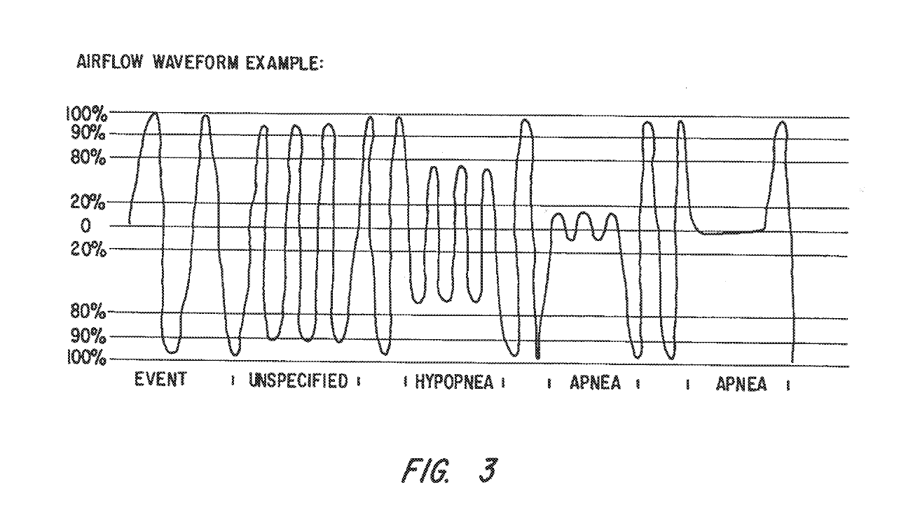

FIG. 3 is an exemplary voltage waveform especially drawn to reflect different sleep disordered breathing events that may be detectable from the airflow waveform analysis algorithm of the present invention. The algorithm is designed to transform the recorded data into the following detected parameters or variables.

Airflow Waveform Analysis Algorithm (Breath to Breath Amplitude Comparison)

Variables:

| Event Start Location | Event End Location | Event Type |
|---|---|---|
| Time Spent In Artifact | | |
| Total Hypopnea Events | Total Apnea Events | Event Log File |

| Time Spent In Apnea | |
|---|---|
| Time Spent In Hypopnea | Time Spent In Unspecified |

| Time Spent Snoring | |
|---|---|
| Snore Start Location | Total Unspecified Events |
| Event Start Location | Total Snore Events |

The algorithms for deriving these parameters from waveforms recorded during a

home screening session will next be explained with reference to FIG. 3 of the drawings.
IDENTIFY BEGINNING OF EVENT

Measure voltage (amplitude) of first breath of recording. If the voltage of the subsequent
breath drops 10% or more as compared to the previous breath, and remains at 10% or
below for more than 2 subsequent breaths, tag the breath just before the first 10% reduced
voltage breath as “Event Start Location” and log the voltage value as “Event Start Voltage”.
Go to Determine Valid Event.

DETERMINE VALID EVENT

Does the voltage for each of the subsequent breaths following the Event Start Location
remain 10% or more below the “Event Start Voltage” for a period of time greater than 10
seconds?

-If yes, it is a valid event - go to “Determine Event End Location”
-If no, abort “Determine Valid Event” routine. Move position to the first breath
following “Event Start Location” that meets or exceeds “Event Start Voltage” and
return to “Identify Beginning of Event”.

DETERMINE EVENT END LOCATION

Does the voltage of the subsequent breaths ever return to, or return to greater than a 10%
reduction of the “Event Start Voltage” within a 120 second timeframe?

-If yes, tag the point of the first breath to reach 10% threshold as “Event End

Location”,

go to Determine Event Type
-If no (exceeds 120 seconds), it becomes an invalid event. Move position to
next breath following the 120 second time frame and return again to
“Identify Beginning of Event”.

DETERMINE EVENT TYPE

Does the voltage of the breaths occurring between “Event Start Location” and “Event End
Location” remain between a 20% - 80% reduction as compared to the “Event Start
Voltage”?

-If yes, the Event Type is Hypopnea. Go to“ Create Report Value”
-If no (greater than 80%), the Event Type is Apnea. Go to “Create Report

Value”

-If no (less than 20%), the Event Type is Unspecified SDB. Go to “Create

Report Value”

-If no (0 volts for more than 90 seconds) the Event Type is Artifact. Continue
and determine total duration until first 2 consecutive breaths to reach 3% or
more of scale. Add duration value to “Time Spent in Artifact”. Move position
to next breath and go to “Identify Beginning of Event”.

CREATE REPORT VALUE

If “Event Type” is Hypopnea:

-Add a value of 1 to “Total Hypopnea Events”
-Calculate duration of event, add duration value to “Time Spent In Hypopnea”
-Append the “Event Log File” with a line:

Event start time (00:00)
“Event Type”
Event duration (seconds)

If “Event Type” is Apnea:

-Add a value of 1 to “Total Apnea Events”
-Calculate duration of event, add duration value to “Time Spent in Apnea”
-Append the “Event Log File” with a line:

Event start time (00:00)
“Event Type”
Event duration (seconds)

If “Event Type” is Unspecified SDB:

-Add a value of 1 to “Total Unspecified Events”
-Calculate duration of event, add duration value to Time Spent in Unspecified
-Append the “Event Log File” with a line:

Event start time (00:00)
“Event Type”
Event duration (seconds)

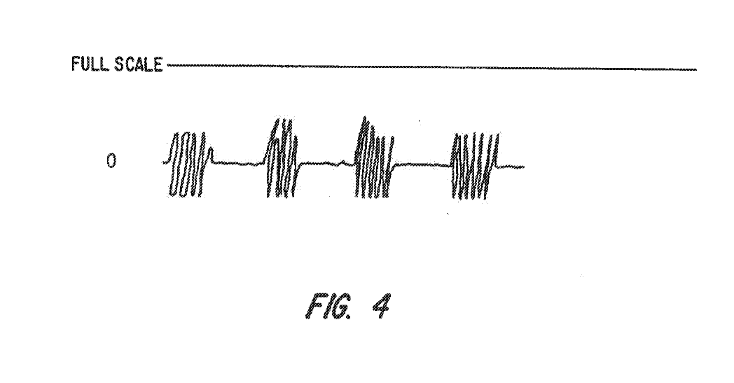

Move position to next breath and return to “Identify Beginning of Event”.
FIG. 4 is a waveform derived from the PVDF film sensor due to vibrational forces due to
snoring which is recorded during the screening phase and subsequently analyzed in
accordance with the following algorithm.

IDENTIFY SNORE START LOCATION

Does a voltage burst within the snore signal exceed 3% of full scale?

-If yes, tag voltage burst as “Snore Start Location”. Add a value of 1 to “Total Snore

Events”

Go to “Determine Snoring Period”

DETERMINE SNORING PERIOD

Is there a subsequent voltage burst meeting the same 3% criteria occurring within a 5
second window relative to the previous voltage burst?

-If yes, add a value of 1 to “Total Snore Events”. Go to Determine Snore Period
-If no, is the “Snore Start Location” value >0?

-If yes, calculate time duration from current “Snore Start Location”, add
value to “Time Spent Snoring”. Maintain current position, “Snore Start
Location”=0, go to “Identify Snoring Start Location”.
-If no, Snore Start Location=0. Maintain current position; go to Identify
Snore Start Location.

Analysis Report Values Creation Algorithm
Once the airflow waveform analysis algorithm has been executed on the host computer
26, a further algorithm is executed on host computer 26 to create a report upon which
recommendations for follow-up treatment can be made. The report values are listed
below and the manner in which each is obtained is explained.

Variables:

| | | | |
|---|---|---|---|
| Event Time | Snore Time | Primary Snore Time | Total Analysis Time |
| Recording Start Time | Recording Stop Time | Total Recording Time | ValidRecordingTime |

| | | |
|---|---|---|
| Valid Analysis Time | Non Event Time | Risk Level |

IDENTIFY RECORDING START TIME

First identifiable breath (voltage burst)=Recording Start Time

IDENTIFY RECORDING STOP TIME

Last identifiable breath=Recording Stop Time

CALCULATE TOTAL RECORDING TIME

Duration from Recording Start Time to Recording Stop Time

DETERMINE VALID RECORDING TIME THRESHOLD

Is Total Recording Time <2 hours?

If yes, Valid Recording Time = Invalid

Abort Analysis Values Creation, Create Invalid Test Report Template

If no, Valid Recording Time = Valid

Proceed with Analysis Values Creation

CALCULATE TOTAL ANALYSIS TIME

Event Time= sum of Time Spent In Apnea +Time Spent In Hypopnea + Time Spent In
Unspecified
Non Event Time=Total Recording Time − Event time − Time Spent in Artifact
Total Analysis Time = Event Time + Non Event Time
Snore Time = Time Spent Snoring
-If Snore Time > Total Analysis Time

Total Analysis Time=Total Analysis Time + (Snore Time−Total Analysis Time)

DETERMINE VALID ANALYSIS TIME THRESHOLD
Is Total Analysis Time <1 hour?

If yes, Valid Analysis Time = Invalid

Abort Analysis Report Values Creation, Create Invalid Test Report Template

If no, Valid Analysis Time = Valid

Proceed with Analysis Values Creation

CALCULATE TOTAL APNEA EVENTS

=Total Apnea Events

CALCULATE TOTAL HYPOPNEA EVENTS

= Total Hypopnea Events

CALCULATE TOTAL UNSPECIFIED SDB EVENTS

= Total Unspecified Events

CALCULATE TOTAL SNORING EVENTS

= Total Snore Events

CALCULATE APNEA INDEX

=Total Apnea Events / Total Analysis Time × 60

CALCULATE HYPOPNEA INDEX

= Total Hypopnea Events / Total Analysis Time × 60

CALCULATE UNSPECIFIED SDB INDEX

= Total Unspecified Events / Total Analysis Time × 60

CALCULATE AHI

=(Total Apnea Events + Total Hypopnea Events) / Total Analysis Time × 60

CALCULATE RDI

=(Total Apnea Events + Total Hypopnea Events + Total Unspecified Events) / Total Analysis

Time × 60
CALCULATE % TIME SPENT IN APNEA

= Time Spent In Apnea / Total Analysis Time

CALCULATE % TIME SPENT IN HYPOPNEA

=Time Spent In Hypopnea / Total Analysis Time

CALCULATE % TIME SPENT IN UNSPECIFIED SDB

= Time Spent In Unspecified / Total Analysis Time

CALCULATE % TIME SPENT SNORING

=Snore Time / Total Analysis Time

EVENT LOG REVIEW (Identify potential artifact or severity)

Review each event entry for a duration exceeding 60 sec.
If duration > 60 sec, highlight log file line with colored text.

DETERMINE RISK LEVEL

If AHI is W or above, Risk Level =X
If RDI is Y or above, Risk Level =Z

The values W, X, Y, Z may be determined at the discretion of a treating physician.

EVENT LOG FILE EXAMPLE:

| | | |
|---|---|---|
| 02:32:18 | Apnea Event | 23 sec |
| 02:34:12 | Apnea Event | 18 sec |
| 02:35:38 | Hypopnea Event | 23 sec |
| 02:36:09 | Apnea Event | 17 sec |
| 02:37:10 | Unspecified Event | 23 sec |
| 02:38:18 | Apnea Event | 64 sec |
| 02:39:55 | Unspecified Event | 20 sec |
| 02:41:00 | Apnea Event | 25 sec |

Based on the AHI being greater than 30, the patient may be directed to immediately obtain and begin using an auto-adjust CPAP system. If the AHI is less than 30, but more than about 10, the patient may be directed to procure a full sleep study.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices. Also, various modifications, both as to the equipment and operating procedures, can be accomplished without departing from the scope of the invention itself.

What is claimed is:

1. A method for screening persons for sleep-disordered breathing comprising the steps of:
(a) providing from a screening test facility to test subjects to be screened a PVDF respiratory air flow sensor and an electronics module which, when coupled to said sensor with the sensor located on a test subject's upper lip, is adapted to record and store signals related to respiratory activity and snoring, said electronics module being pre-loaded with a given test subject's demographic information;
(b) obtaining a return to the screening test facility of the electronics module following a period of sleep in which the sensor and electronics module are being worn by said given test subject;
(c) downloading from the returned electronics module into a host computer at the screening test facility the given test subject's demographic information and information related to respiratory activity and snoring that had been derived from the sensor and stored;
(d) executing a program on the host computer for analyzing the sensor-derived information to identify the onset, duration and type of sleep-disordered breathing events; and
(e) separately logging the type of sleep-disordered breathing events.

2. The method of claim 1 wherein the electronics module includes:
(a) an amplifying and filtering circuit for producing a first analog signal relating to respiratory air flow impinging on the sensor and second analog signals related to snoring derived from the sensor;
(b) an analog to digital converter connected to receive the first and second analog signals; and
(c) a memory for recording outputs from the analog to digital converter.

3. The method of claim 1 and further including the step of preparing in the host computer a report identifying total apnea events, total hypopnea events, total unspecified sleep-disordered breathing events and total snoring events during a calculated total analysis time.

4. The method of claim 1 and further including the steps of defining an onset of a respiratory event by measuring a signal amplitude of an initial breath and detecting whether the signal amplitude of a following breath drops 10 percent or more from the initial breath and remains at the reduced amplitude for two or more subsequent breaths and identifying the Event Start Location and Event Start Voltage as a point just prior to the signal amplitude drop.

5. The method of claim 4 and further comprising the step of detecting whether the amplitude of each breath following the Event Start Location remains at 10 percent or more below the Event Start Voltage at least for 10 seconds and whether subsequent breath signal amplitudes ever return to a value greater than the 10 percent drop within a 120 second time frame to define an Event End Location as the first breath signal amplitude excursion back above the 10 percent drop level.

6. The method of claim 5 and further comprising the step of:
determining whether the voltage amplitude of breaths occurring between the Event Start Voltage and the Event End Location stay between a 20 percent to 80 percent reduction to identify an hypopnea event, and if greater than 80 percent reduction, to identify an apnea event, and if the voltage amplitude of breaths occurring between the Event Start Location and the Event End Location remain above a 20 percent reduction to identify an unspecified sleep-disordered breathing.

7. A method for home screening persons to assess degree of possible sleep-disorder breathing comprising the steps of:
(a) providing from a screening test facility to a given subject a PVDF pyro/piezo transducer adapted for placement on a person's upper lip in the path of respiratory air flow and an electronics module adapted to be coupled to said transducer, said electronics module comprising a signal processing circuit for amplifying, filtering and separating transducer output signal trains into first and second channels, the first channel providing analog signals proportional to temperature shifts due to impingement of inspiratory and expiratory air flow on said PVDF transducer and the second channel providing analog signals related to snoring, said electronics module further comprising an analog-to-digital converter coupled to receive the analog signals from the first and second channels, and a microprocessor with a memory for storing outputs from the analog-to-digital converter;
(b) prior to step (a), entering from a host computer at the screening test facility into the memory of the electronics module demographic data of said given test subject;
(c) instructing the given test subject prior to his retiring for sleep how to append the PVDF transducer to his or her upper lip and how to couple the PVDF transducer to the electronics module;
(d) following a period of sleep of at least two hours, obtaining a return of the electronics module to the screening test facility; and
(e) downloading the contents of the memory into a hose computer and executing a program in the host computer for analyzing information derived from the test subject during the period of sleep to identify the occurrence, frequency and time of events of sleep-disordered breathing and snoring.

8. The method of claim 7 wherein the events identified from executing the program include hypopnea, apnea, unspecified sleep-disordered breathing and snoring.

9. The method of claim 7 and further including the step of deriving a apnea hypopnea index from the information derived from the test subject during the period of sleep.

10. The method of claim 9 and further providing a recommended treatment modality based upon the derived apnea hypopnea index.
